(19) United States
(12) Patent Application Publication
(10) Pub. No.: US 2015/0336999 A1
(43) Pub. Date: Nov. 26, 2015

(54) PROCESS FOR THE PRODUCTION OF SOPHOROSE STARTING FROM SOPHOROLIPIDS

(71) Applicant: IFP ENERGIES NOUVELLES, Rueil-Malmaison (FR)

(72) Inventors: Etienne JOURDIER, Bougival (FR); Fadhel BEN CHAABANE, Paris (FR)

(73) Assignee: IFP Energies nouvelles, Rueil-Malmaison Cedex (FR)

(21) Appl. No.: 14/438,988

(22) PCT Filed: Sep. 26, 2013
(86) PCT No.: PCT/FR2013/052269

§ 371 Date: Apr. 28, 2015

(30) Foreign Application Priority Data
Oct. 29, 2012 (FR) 12/02892

(51) Int. Cl. C07H 3/04; C07H 1/06; C07H 1/00

(52) U.S. Cl. CPC C07H 3/04; C07H 1/00; C07H 1/06

(57) ABSTRACT

The present invention concerns a process for the production of sophorose starting from sophorolipid, in which a step for the hydrolysis of sophorolipid is carried out in an aqueous medium in the presence of an acidic compound.

The present invention relates to a process for the production of sophorose by acid hydrolysis of sophorolipids. The invention also relates to the use of a hydrolysate obtained using the process of the invention as a solution for inducing the expression of genes coding for cellulases. Finally, the invention relates to a process for the purification of sophorose starting from the hydrolysate.

### PRIOR ART

Cellulases adapted to the hydrolysis of polysaccharides allow the microorganisms which produce them to use cellulose and hemicellulose (the major components of plants) as a source of carbon, by hydrolysing these polymers into simple sugars (glucose, xylose, etc.).

Currently, these enzymes are employed in many fields such as detergents, the textile industry or the food industry, as well as in processes aimed at the biochemical upgrading of lignocellulosic biomass (for example for the production of second generation ethanol biofuel). Trichoderma reesei is currently recognized as the best cellulase-producing microorganism in processes for the production of biofuels starting from lignocellulosic biomass, principally because of its high secreting ability.

However, the production cost for cellulases remains high and represents one of the economic obstacles to the industrial scale application of processes known as second generation processes.

Industrially, the optimized production of cellulases by Trichoderma reesei is obtained in a fed-batch protocol (supply without withdrawal) using a feed solution containing lactose as the sugar inducing the production of cellulases (EP 0 448 430 B1). Although it is used on an industrial scale, lactose has the disadvantage of being expensive having regard to the production of low cost cellulases.

Sophorose is known to be a very good inducer of the cellulolytic Trichoderma reesei system. In 1962, Mandels M. et al. [Sophorose as an inducer of cellulase in Trichoderma viride. Journal of Bacteriology 1962, 83:400-408] demonstrated that, although only present in a quantity of 0.006% as an impurity in glucose, sophorose could be used to induce the production of cellulolytic enzymes (or cellulases). These sophorose impurities were formed by a mechanism of transglycosylation during the acid hydrolysis of starch to glucose.

Sophorose (2-O-β-D-glucopyranosyl-D-glucose) is a diholoside constituted by two glucose units connected via an osidic bond β(1→2). It is present in nature, for example in cloves from the Sophora japonica tree, from which it was isolated for the first time in 1938.

Unfortunately, the high price of sophorose means that it is still not possible to use it on an industrial scale. Until now, it has only been used in the laboratory for experiments necessitating strong induction.

One aim of the present invention is to overcome the disadvantages described above, and in particular to propose a simple and economical process for the production of sophorose starting from sophorolipids. The sophorose contained in the hydrolysate may then be used as an inducing agent in microorganisms capable of producing cellulases, in particular in Trichoderma reesei.

### SUMMARY OF THE INVENTION

This aim is accomplished by means of a process in which:

a) an aqueous solution of sophorolipids is provided;

b) an acidic compound is added to the aqueous solution of sophorolipids, the quantity of the acidic compound being in the range 0.01% to 6% by weight with respect to the weight of sophorolipids;

c) the mixture obtained in step b) is heated for 2 to 200 hours and at a temperature in the range 60° C. to 110° C.

The process of the invention employs a hydrolysis of sophorolipid in an aqueous medium in the presence of an acidic compound, i.e. which is capable of liberating H⁺ ions in solution.

In the context of the present invention, the term “sophorolipid” (or sophorose lipid) denotes compounds comprising a sophorose unit connected to a fatty acid via an osidic type bond (between the 1′ carbon of the sophorose and the (ω-1 carbon of the fatty acid). The sophorolipids may be produced by microorganisms, in particular by yeasts of the genus Candida. Depending on the lipid supplied to the microorganism and the culture conditions, a mixture of different sophorolipids may be obtained, in the acidic form (free carboxylic function) or cyclic lactone form (ester bond between the fatty acid and the —OH group in the 4″ position of sophorose), and with the 6′ and 6″ —OH groups which may be acetylated.

The quantity of sophorolipids in the medium is generally in the range 2 to 800 g/L, and preferably in the range 100 to 400 g/L.

The quantity of the acidic compound is in the range 0.01% to 6% by weight with respect to the weight of sophorolipids, preferably in the range 0.1% to 2% by weight with respect to the weight of sophorolipids. Still more preferably, the quantity of the acidic compound is in the range 0.1% to 1% by weight with respect to the weight of sophorolipids.

In accordance with a particular embodiment, the quantity of sophorolipid in the medium is in the range 100 to 400 g/L and the quantity of acidic compound is in the range 0.1% to 2% by weight with respect to the weight of sophorolipids.

The acidic compound employed is an inorganic acid or an organic acid.

Preferably, the acidic compound is an inorganic acid selected from sulphuric acid, hydrochloric acid, phosphoric acid and nitric acid. Preferably, the inorganic acid is sulphuric acid.

In accordance with a particular embodiment of the process, a step for separation of the lipid phase and the aqueous phase of the mixture obtained from the hydrolysis step is carried out in order to recover an aqueous solution (hydrolysate) containing sophorose and glucose.

The hydrolysate obtained in this manner at the end of the hydrolysis step and after separation of the lipid phase is used as a solution to induce the expression of genes coding for cellulases in a microorganism such as Trichoderma reesei, for example.

Before using it as an inducing solution, it is also possible to purify said aqueous solution containing the mixture of sophorose and glucose by adding an alcoholigenic microorganism which is capable of fermenting the glucose to form ethanol to this solution, then by carrying out an ethanolic fermentation and finally by separating out the ethanol (for example by stripping) in order to recover a purified hydrolysate containing sophorose.

### DETAILED DESCRIPTION OF THE INVENTION

The present invention describes the conditions for carrying out an acid hydrolysis with the aim of producing sophorose and avoiding total hydrolysis of the sophorolipids, which would result in the liberation of two glucose units per molecule of sophorolipid.

Under the conditions described below, the hydrolysis can be used to preferentially rupture the osidic bond between the sophorose and the lipid chain, which results in the liberation of sophorose. However, inevitably and to a greater or lesser extent depending on the operating conditions, the sophorose which is liberated may itself also be hydrolysed into two glucose units.

In practice, initially, an aqueous solution of sophorolipids is provided. This aqueous solution typically has a concentration of sophorolipids in the range 2 to 800 g/L, preferably in the range 100 to 400 g/L. Preferably, sophorolipids will be used in the acidic form rather than in the lactonic form. The acidic form may be obtained either by optimizing the fermentation conditions during the production of sophorolipids, or by carrying out a hydrolysis of the fermentation product under basic conditions (saponification).

An acidic compound is added to this solution in order to produce a concentration of acid in the mixture in the range 0.01% to 6% by weight with respect to the weight of sophorolipids, preferably in the range 0.1% to 2% by weight and more preferably in the range 0.1% to 1% by weight.

The acidic compound is either an organic acid or an inorganic acid. Preferably, an inorganic acid is used which may be selected from sulphuric acid, hydrochloric acid, phosphoric acid and nitric acid. Preferably, the acid is sulphuric acid. If an organic acid is used it may be selected from formic acid, acetic acid, oxalic acid, trifluoroacetic acid and maleic acid.

The hydrolysis is carried out by heating the acid mixture to a temperature which is generally in the range 60° C. to 110° C. for 2 hours to 200 hours, preferably in the range 5 hours to 24 hours. At the end of this treatment, the fatty acids liberated by the hydrolysis form a phase which is not miscible with water, which can be readily separated from the aqueous phase. The aqueous phase (termed the hydrolysate) contains a mixture of glucose and sophorose the proportions of which vary as a function of the dose of acid used and the period of hydrolysis.

The hydrolysate obtained in this manner may be used for the production of proteins of interest (cellulases) by microorganisms the coding genes of which are under the control of a promoter which is inducible by the sophorose. In particular, the microorganism is from the genus Trichoderma, preferably Trichoderma reesei.

Any type of production process may be used which is suitable for the microorganism and known to the skilled person. In particular, the hydrolysate containing the sophorose is employed in a process of the “fed-batch” type, as described in the document FR 2 555 603.

To this end, a preculture is prepared containing the microorganism and a culture medium containing mineral salts and the usual vitamin supplements and a source of carbon and energy, preferably in the form of soluble sugars (for example lactose). The preculture is then transferred to an enzyme production fermenter which comprises a culture medium containing at least one sugar and supplemented with an inducing solution in the usual manner. In the context of the invention, the inducing solution used is the hydrolysate obtained from sophorolipid hydrolysis. Next, the enzymes are produced in the fermenter by maintaining the necessary contact between the microorganism and the culture medium and by supplying the sophorose-inducing solution in a regular manner (for example continuously).

Before use, it is also possible to purify the mixture (hydrolysate) obtained from the hydrolysis step in order to isolate the sophorose produced. To this end, a microorganism is added to said mixture which is capable of fermenting glucose into ethanol, such as the yeast Saccharomyces cerevisiae, for example, and then a fermentation step is carried out. The final mixture obtained after fermentation undergoes a step for stripping the ethanol in order to recover an aqueous solution of sophorose. It is also possible to use the aqueous solution of sophorose obtained as a solution for inducing the expression of genes in the microorganisms.

### DESCRIPTION OF THE FIGURES

FIG. 1 represents the concentration of cellulases produced in the presence of glucose, lactose and sophorolipid, alone or as a mixture.

FIG. 2 represents the change in the concentrations of glucose and sophorose as a function of the concentration of sulphuric acid employed for the controlled hydrolysis of the sophorolipids.

FIG. 3 compares the specific rates of cellulase production by Trichoderma reesei during a production with a fed-batch supply using a solution comprising glucose and lactose or sophorose.

### EXAMPLES

### Example 1 (Not In Accordance With The Invention)

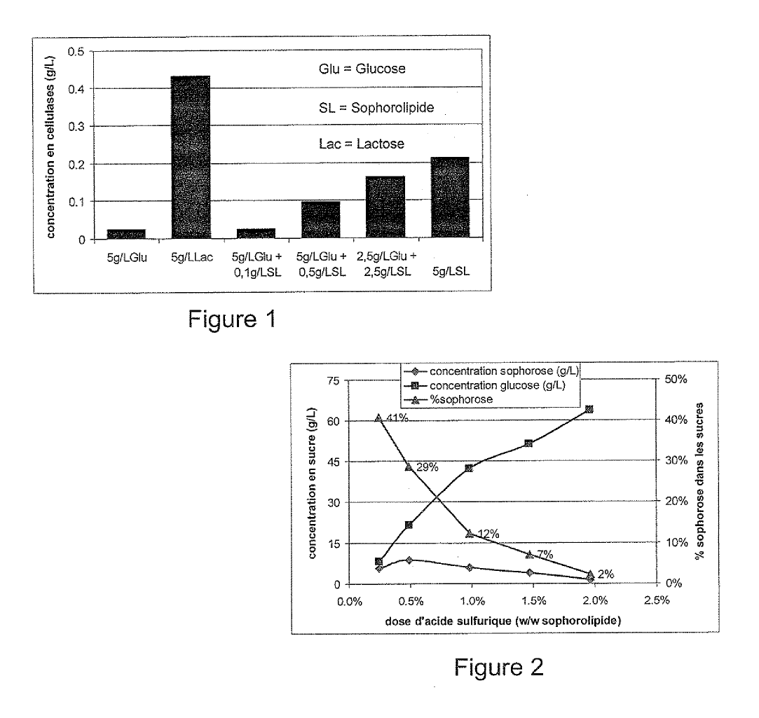

After growing T. reesei in a flask on 10 g/L of glucose and until exhaustion of the glucose, a medium was obtained containing 5 g/L of biomass (T. reesei cells). In this medium, injections of concentrated glucose solutions and sophorolipid solutions in the acidic form obtained from a Candida bombicola culture were carried out in order to obtain the desired quantities of glucose and sophorolipid as indicated in FIG. 1. Injections of glucose alone, lactose alone (conventional industrial inducer) and sophorolipid alone were also carried out, corresponding to control media.

After 20 h of incubation, the quantity of proteins (cellulases) produced in the various test media was analysed. The protein assay was carried out using the method by Lowry OH et al. (1951) [Protein measurement with the Folin phenol reagent. Journal of Biological Chemistry 193, 1, 265-275] using the DC Protein Assay kit (Biorad).

Referring now to FIG. 1, the results show that the quantity of enzymes produced in the presence of sophorolipids is still lower than in the presence of lactose, and thus that the unrefined sophorolipid is a poorer inducer than lactose.

### Example 2 (Optimization Of Conditions For Controlled Acid Hydrolysis Of Sophorolipids)

In test tubes, 1 mL of an aqueous sophorolipid solution (obtained from a Candida bombicola culture) in the acidic form, approximately 250 g/L, and concentrated sulphuric acid were mixed in order to obtain a concentration of sulphuric acid in the range 0.25% to 2% by weight of acid with respect to the weight of sophorolipid.

The test tubes were sealed with a hermetic stopper, then placed in a dry bath at 100° C. for 16 hours. The concentrations of sugars (glucose and sophorose) liberated during the hydrolysis were measured using HPLC.

Referring to FIG. 2, it can be seen that when the quantity of sulphuric acid was increased, the total liberation of sugars was improved (for the same hydrolysis period), however the mixture contained mainly glucose. In contrast, for the smaller quantities of sulphuric acid, i.e. in the range 0.1% to 1% by weight of sulphuric acid with respect to the weight of sophorolipid, after hydrolysis, the total sugar content of the mixture was lower, but the proportion of sophorose was much higher (41% by weight of sophorose in the mixture hydrolysed with 025% by weight of sulphuric acid with respect to the weight of sophorolipids).

An identical experiment carried out for 5 hours with 8% by weight of sulphuric acid with respect to the weight of sophorolipids caused the liberation of glucose alone without the associated liberation of sophorose.

### Example 3 (Controlled Sophorolipid Hydrolysis)

200 mL of a solution containing approximately 200 g/L of sophorolipids in the acidic form and 0.25% of sulphuric acid with respect to the weight of sophorolipid was prepared. This solution was set to boil in a retort, with stirring and under reflux, for 12 hours. The oily fraction containing the lipids resulting from hydrolysis was separated from the aqueous phase and the sugars (glucose and sophorose) liberated into the aqueous fraction were assayed by HPLC. The concentrations measured were 10.3 g/L of sophorose and 16.6 g/L of glucose, i.e. 38% by weight of sophorose in the liberated mixture of sugars.

### Example 4 (Use Of Sophorolipid Hydrolysate To Induce The Production Of Cellulases By T. reesei)

After growing cells of T. reesei CL847 (Durand H. et al. (1988a) Classical and molecular genetics applied to Trichoderma reesei for the selection of improved cellulolytic industrial strains. In: FEMS Symposium n° 43: Biochemistry and Genetics of Cellulose Degradation, p. 135-151. Academic Press, London) on glucose in stirred flasks up to the exhaustion of the glucose, cellulase production was carried out using a “fed-batch” process employing a solution the composition of which was as follows: 50 g/L of a mixture of sugars and 15 mL/L of 20% by weight NH₃ as described in the publication “A new stoichiometric miniaturization strategy for screening of industrial microbial strains: application to cellulase hyper-producing Trichoderma reesei strains”, Microbial Cell Factories 2012, 11:70.

The mixture of sugars used contained (100−x)% by weight of glucose and x% by weight of inducing sugar:

in the first case (control), the inducing sugar was lactose at a quantity of x=8%

in the second, third and fourth cases, the inducing sugar was sophorose from the hydrolysate obtained in Example 3 which had been supplemented with pure glucose in order to have x=8%, x=0.3% and x=0.03% respectively.

The fed-batch process was carried out for 166 hours with a solution supply flow rate of 0.3 mL/h and at a temperature of 30° C.

Daily samples were taken in order to monitor the concentration of cells and the concentration of proteins. The induction capacity of the mixture of sugars used for the fed-batch process was expressed as the specific rate of protein production (mg of proteins produced per gram of producer cells per hour).

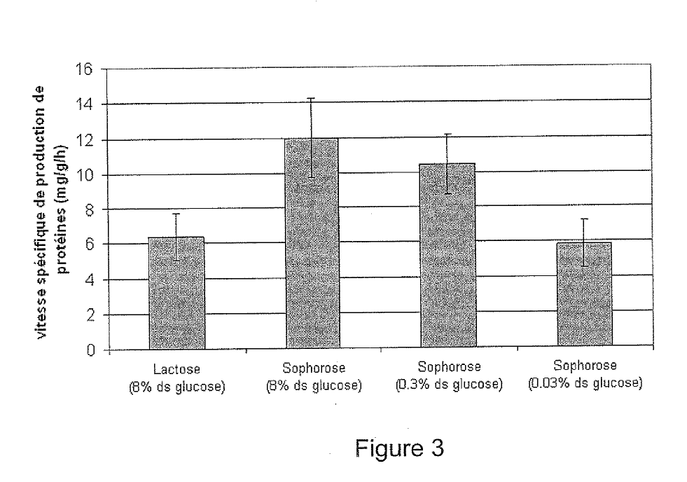

It will be observed in FIG. 3 that the mixture containing sophorose caused an induction approximately twice as high as the mixture containing lactose at the same concentration, respectively with specific protein production rates of 12 as opposed to 6.4 mg/g/h.

In addition, it will be observed in FIG. 3 that the mixture containing 0.03% of sophorose already caused an induction equivalent to the mixture containing 8% of lactose, with specific protein production rates of the order of 6 mg/g/h.

Finally, it will be observed in FIG. 3 that the mixture containing 0.3% of sophorose caused an induction equivalent to the mixture containing 8% of sophorose, with respective specific protein production rates of 10.5 and 12 mg/g/h.

1. A process for the production of sophorose starting from sophorolipids, in which:
a) an aqueous solution of sophorolipids is provided;
b) an acidic compound is added to the aqueous solution of sophorolipids, the quantity of the acidic compound being in the range 0.01% to 6% by weight with respect to the weight of sophorolipids;
c) the mixture obtained in step b) is heated for 2 to 200 hours and at a temperature in the range 60° C. to 110° C.

2. The process according to claim 1, in which the quantity of sophorolipids in the aqueous solution is in the range 2 to 800 g/L.

3. The process according to claim 1, in which the quantity of acidic compound is in the range 0.1% to 2% by weight with respect to the weight of sophorolipids.

4. The process according to claim 1, in which the acidic compound is an inorganic acid or an organic acid.

5. The process according to claim 4, in which the inorganic acid is selected from sulphuric acid, hydrochloric acid, phosphoric acid and nitric acid.

6. The process according to claim 1, in which a step for separation of the lipid phase and the aqueous phase of the mixture obtained from step c) is carried out in order to recover an aqueous hydrolysate containing sophorose and glucose.

7. A process for the purification of sophorose from a hydrolysate obtained according to the process of claim 6, comprising the following steps:
a) adding a microorganism which is capable of fermenting the glucose into ethanol in the hydrolysate;
b) carrying out an ethanolic fermentation of the hydrolysate in order to provide a fermented hydrolysate;
c) stripping the ethanol present in the fermented hydrolysate.
